(19) United States
(12) Patent Application Publication
(10) Pub. No.: US 2026/0224921 A1
(43) Pub. Date: Aug. 6, 2026

(54) INTRACAVITARY ULTRASOUND DEVICE EQUIPPED WITH A LINEARLY MOVABLE ULTRASOUND PROBE

(71) Applicant: KORUST CO., LTD., Anyang-si, Gyeonggi-do (KR)

(72) Inventors: Sung-Chan CHO, Seongnam-si (KR); Yoon Oh Nam, Siheung-si (KR)

(73) Assignee: KORUST CO., LTD., Anyang-si, Gyeonggi-do (KR)

(21) Appl. No.: 19/153,384

(22) PCT Filed: Nov. 16, 2023
(86) PCT No.: PCT/KR2023/018453

§ 371 Date: Aug. 4, 2025

(30) Foreign Application Priority Data
Feb. 7, 2023 (KR) 10-2023-0016324

(51) Int. Cl. A61N 7/02

(52) U.S. Cl. CPC A61N 7/022

(57) ABSTRACT

An intracavitary ultrasound device includes a support bar extending in the longitudinal direction, an ultrasound probe supported by the support bar and configured to be insertable into a body cavity, a sealing cover configured to surround at least a portion of the ultrasound probe and the support bar, and an ultrasound transmission medium supply tube configured to supply an ultrasound transmission medium to a space where the ultrasound probe is disposed. The ultrasound transmission medium supply tube is configured to supply the ultrasound transmission medium past the ultrasound probe to a distal space of the sealing cover.

### TECHNICAL FIELD

The present invention relates to an intracavitary (i.e., body cavity-insertable) ultrasound device configured to be inserted into a body cavity such as the nasal cavity, oral cavity, pharynx, vagina, or urethra of a human body to perform ultrasound procedures.

### BACKGROUND ART

Ultrasound has been used for various treatments and procedures; for example, a method has been introduced in which ultrasound is non-invasively irradiated within a body cavity, such as the nasal cavity, to treat diseases using heat.

In general, an ultrasound device using focused ultrasound includes an ultrasound probe and a pulse power generator. The ultrasound probe comprises a piezoelectric element that generates ultrasound and a housing that secures the piezoelectric element, which is supported by a support bar extending from a handle. Additionally, a sealing cover enclosing the ultrasound probe is provided, and the sealing cover is filled with an ultrasound transmission medium such as water. The transmission medium contained within the sealing cover serves not only to transmit ultrasound to the human body, but also to cool the heat generated by the piezoelectric element during ultrasound generation. Therefore, if necessary, the ultrasound transmission medium should be capable of circulating to perform effective cooling.

Since the ultrasound probe enclosed by the sealing cover must be small enough to be inserted into a body cavity, both the sealing cover and the ultrasound probe must have compact dimensions, but there is a limit to reducing the size of the piezoelectric element required to generate sufficient ultrasound energy for therapeutic effects and consequently, the size of the ultrasound probe that includes the piezoelectric element also cannot be significantly reduced, resulting in a narrow space between the sealing cover and the probe. This makes it difficult to circulate the ultrasound transmission medium. Furthermore, if the ultrasound probe is required to reach deeper areas within the body cavity, the probe must be elongated, which further complicates the circulation of the transmission medium.

Since reducing the size of the ultrasound probe leads to a decrease in ultrasound energy, there is a limit to how small the probe can be, especially when the diameter of the sealing cover is small. As a result, it becomes difficult to ensure sufficient space between the outer peripheral surface of the ultrasound probe and the inner peripheral surface of the sealing cover for the ultrasound transmission medium to flow. Consequently, it becomes increasingly difficult for the transmission medium supplied from the handle side to fill the space between the distal end of the sealing cover and the ultrasound probe, and it is also difficult for the medium to be discharged. This problem is further exacerbated when the ultrasound probe is long, making it even more difficult for the medium delivered into the space between the probe and the sealing cover to be discharged.

Furthermore, in the case of a structure in which the sealing cover is separable and attachable to the handle, air tends to remain inside the sealing cover during the attachment process. Due to the pressure of this residual air, it becomes difficult to fully fill the interior space of the sealing cover with the ultrasound transmission medium, even when the medium is supplied.

If the sealing cover is not sufficiently filled with the ultrasound transmission medium, ultrasound may not be properly transmitted, or the heat generated by the piezoelectric element may not be adequately dissipated, which could result in damage to the piezoelectric element.

Prior Art Document: U.S. Patent Application Publication No. US2008/0027423

### DETAILED DESCRIPTION OF THE INVENTION

### Technical Problem

The object of the present invention is to provide a solution that allows the ultrasound transmission medium to be easily filled within a sealing cover having a narrow and elongated structure, and in particular, to ensure that the transmission medium can be effectively filled to the distal end of the sealing cover even when the ultrasound probe has a structure that enables linear movement within the cover.

### Technical Solutions

According to an embodiment of the present invention, an intracavitary ultrasound device includes: a support bar extending in a longitudinal direction; an ultrasound probe supported by the support bar and configured to be insertable into a body cavity; a sealing cover configured to surround at least a portion of the ultrasound probe and the support bar; and an ultrasound transmission medium supply tube configured to supply an ultrasound transmission medium to a space in which the ultrasound probe is disposed. The ultrasound transmission medium supply tube is configured to supply the ultrasound transmission medium past the ultrasound probe to a distal space of the sealing cover.

The ultrasound probe may include a housing and an ultrasonic piezoelectric element fixed to the housing, and the ultrasound transmission medium supply tube may be installed such that its distal end extends past the housing and is exposed to an inner space of the sealing cover.

The housing may be provided with a recessed groove, and the ultrasound transmission medium supply tube may be inserted into the recessed groove.

According to another embodiment of the present invention, an intracavitary ultrasound device may further include a fastening member that couples the housing and the ultrasound transmission medium supply tube to each other so as to prevent the ultrasound transmission medium supply tube from disengaging from the recessed groove of the housing.

The fastening member may be configured to couple the housing and the ultrasound transmission medium supply tube to each other in a manner that allows relative movement between the housing and the ultrasound transmission medium supply tube, while preventing the ultrasound transmission medium from disengaging from the housing.

The ultrasound probe may be disposed within the sealing cover in a state allowing linear movement.

The ultrasound probe may be configured to move linearly together with the support bar.

The ultrasound transmission medium supply tube may be configured to serve as a guide for the linear movement of the ultrasound probe while maintaining a predetermined position during the linear movement of the ultrasound probe.

According to another embodiment of the present invention, an intracavitary ultrasound device may further include an ultrasound transmission medium discharge tube configured to discharge the ultrasound transmission medium from the space in which the ultrasound probe is disposed.

The ultrasound probe may include a housing and an ultrasonic piezoelectric element fixed to the housing. The housing may be provided with a pair of recessed grooves, and the ultrasound transmission medium supply tube and the ultrasound transmission medium discharge tube may be respectively inserted into the pair of recessed grooves.

According to another embodiment of the present invention, an intracavitary ultrasound device may further include a handle to which the support bar is connected, and the ultrasound probe and the support bar may be configured to move relative to the handle.

The ultrasound transmission medium supplied into the sealing cover through the ultrasound transmission medium supply tube may be configured to be discharged to the outside through either the interior or the exterior of the handle.

According to another embodiment of the present invention, the sealing cover may be provided with an ultrasound transmission medium inlet passage for introducing the ultrasound transmission medium, and an ultrasound transmission medium outlet passage for discharging the ultrasound transmission medium.

The intracavitary ultrasound device may further include a handle to which the support bar is connected, and the sealing cover may be configured to be detachable from the handle.

### Effect of the Invention

According to the present invention, the ultrasound transmission medium can be smoothly supplied to the space at the distal ends of the ultrasound probe and the sealing cover, thereby ensuring stable ultrasound propagation and excellent cooling performance.

### BRIEF DESCRIPTION OF THE DRAWINGS

FIG. 1 is a perspective view of an intracavitary ultrasound device according to an embodiment of the present invention.

FIG. 2 is a partially exploded perspective view of an intracavitary ultrasound device according to an embodiment of the present invention.

FIG. 3 is a cross-sectional view of an intracavitary ultrasound device according to an embodiment of the present invention.

FIG. 4 is a perspective view showing the state in which the case of the handle and the sealing cover are removed from the intracavitary ultrasound device according to an embodiment of the present invention.

FIG. 5 is a sectional view taken along line A-A of FIG. 1.

FIG. 6 is a perspective view of the ultrasound probe of an intracavitary ultrasound device according to an embodiment of the present invention.

FIG. 7 is a view showing the coupling state of the ultrasound probe and the water supply tube of the intracavitary ultrasound device according to an embodiment of the present invention.

FIG. 8 is a view showing a state in which the ultrasound probe of the an intracavitary ultrasound device has moved backward relative to the water supply tube according to an embodiment of the present invention.

FIG. 9 is a cross-sectional view of the ultrasound probe, the water supply tube, and the drainage tube of the intracavitary ultrasound device according to another embodiment of the present invention.

FIG. 10 is a view showing a sealing cover of the intracavitary ultrasound device provided with separate transmission medium inflow and outflow passages and a water supply tube according to yet another embodiment of the present invention.

### BEST MODE FOR CARRYING OUT THE INVENTION

Hereinafter, embodiments of the present invention will be described in detail with reference to the accompanying drawings. The following detailed description is provided to enable those skilled in the art to which the present invention pertains to readily implement the invention. However, the present invention may be embodied in various different forms and should not be construed as limited to the embodiments described herein.

In describing the components of the present invention, terms such as first, second, A, B, (a), and (b) may be used. These terms are used merely to distinguish one component from another and do not imply any intrinsic characteristics, order, or priority of the components. When a component is described as being “connected”, “coupled”, or “joined” to another component, it should be understood that the component may be directly connected, coupled, or joined to the other component, or it may be indirectly connected, coupled, or joined via another component.

An intracavitary (i.e., body cavity-insertable) ultrasound device according to an embodiment of the present invention is configured to emit ultrasound while being inserted into a body cavity such as the oral cavity, nasal cavity, pharynx, vagina, or urethra, thereby enabling ultrasound-based procedures, for example, procedures that induce thermal lesions in tissue using ultrasound energy.

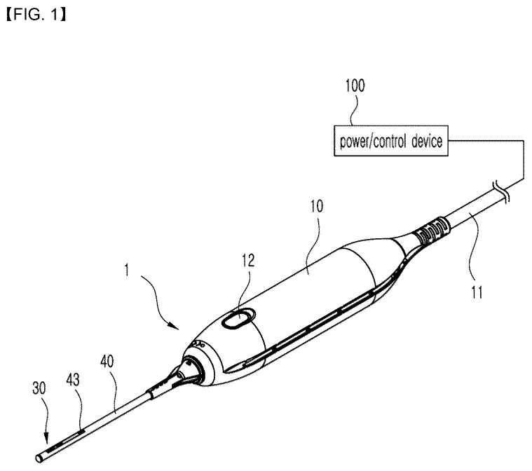

Referring to FIG. 1, an intracavitary ultrasound device 1 according to an embodiment of the present invention includes a handle 10, a support bar 20, an ultrasound probe 30, and a sealing cover 40. The ultrasound probe 30, which is supported by the support bar 20, is configured such that at least a portion of the ultrasound probe 30 and the support bar 20, namely the distal end portion, has a size and shape suitable for insertion into a body cavity.

The handle 10 is formed to be grasped by a user performing an ultrasound procedure. As illustrated by way of example in FIG. 1, the handle 10 is configured to be connected to an external power/control unit 100 via a power connection line 11, and may include an on/off button 12 for controlling ultrasound generation based on the power supply status. The power/control unit 100 may be configured to supply pulse power for ultrasound generation and perform various controls for the ultrasound procedure. The intracavitary ultrasound device 1 may implement the controller for controlling the operation of the ultrasound probe 30 through the power/control unit 100.

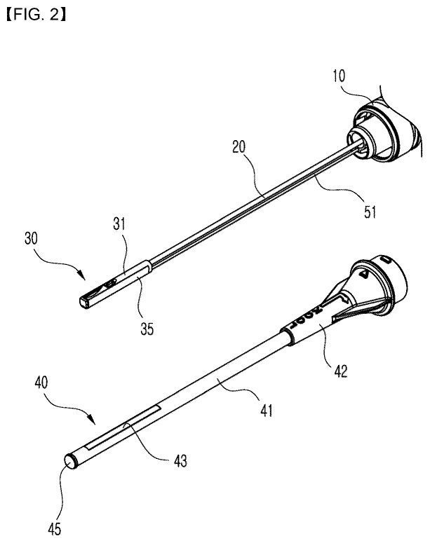

As shown in FIG. 2, the support bar 20 extends from the distal end of the handle 10 and may have a rod shape or a pipe shape. Since the distal end of the support bar 20 may be inserted into a body cavity during the ultrasound procedure, the support bar 20 may be formed as a rod having a relatively small diameter suitable for insertion into the body cavity. In another embodiment of the present invention, the support bar 20 may be connected to a separate support structure instead of the handle.

The support bar 20 extending from the handle 10 is sealed by the sealing cover 40, and the interior of the sealing cover 40 may be filled with an ultrasound transmission medium to enable the transmission of ultrasound generated by the ultrasound probe 30. The sealing cover 40 surrounds the support bar 20 and the ultrasound probe 30 supported thereby, and functions to enclose the ultrasound transmission medium for ultrasound propagation. The ultrasound transmission medium not only serves as a medium for transmitting ultrasound, but may also perform a cooling function by absorbing heat generated during operation of the ultrasound probe 30. The structure for supplying and circulating the ultrasound transmission medium will be described below.

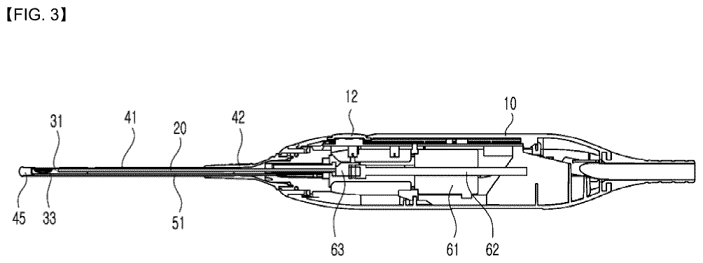

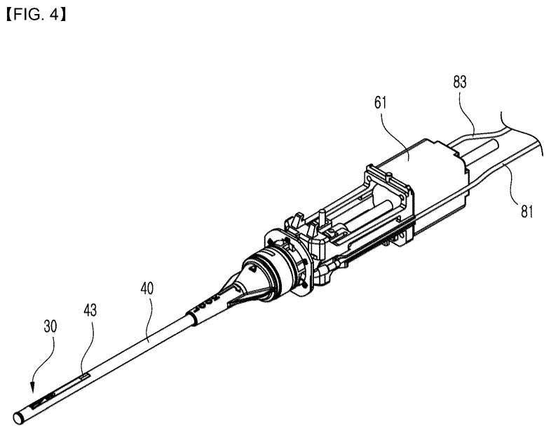

The ultrasound probe 30 generates ultrasound vibrations by the application of pulse power. Referring to FIGS. 2 and 3, the ultrasound probe 30 includes an ultrasound piezoelectric element housing 31 and an ultrasound piezoelectric element 33. The ultrasound piezoelectric element 33 may be composed of a piezoelectric material layer, such as a piezoelectric ceramic, and a pair of electrodes formed on both surfaces of the layer, as is conventionally known, and when a pulse power is applied to both electrodes, the piezoelectric material layer is configured to generate ultrasound vibrations. Although not shown in the drawings, a power line for applying pulse power to the ultrasound piezoelectric element 33 may be provided, and for example, the power line may pass through the handle 10 and the support bar 20 and be electrically connected to both electrodes of the ultrasound piezoelectric element 33. For instance, the support bar 20 may have a hollow structure with a through-hole, and the power line may be routed through the through-hole to be connected to the ultrasound piezoelectric element 33.

The ultrasound piezoelectric element 33 may be configured to focus ultrasound U at a desired location. For example, the ultrasound piezoelectric element 33 may be formed in various shapes capable of generating focused ultrasound, such as a concave curved surface, a concave cylindrical surface, a spherical surface, or a spherical surface with portions removed from both sides.

The ultrasound probe 30 is supported by the support bar 20. For example, the housing 31 of the ultrasound probe 30 may be fixed to the distal end of the support bar 20, whereby the ultrasound probe 30 is supported by the support bar 20.

The sealing cover 40 for accommodating the ultrasound probe 30 is provided. The sealing cover 40 may be fastened to the handle 10 in a state of surrounding the support bar 20 and the ultrasound probe 30. The sealing cover 40 may be configured to be detachable from the handle 10. Referring to FIGS. 1 and 2, the sealing cover 40 may have the form of a hollow pipe having an overall elongated rod-like shape, similar to the support bar 20. For example, the sealing cover 40 may include a tubular member 41, a fastening member 42 fastened to one end of the tubular member 41 and configured to be coupled to the handle 10, and an end cap 45 fastened to the distal end of the tubular member 41. In another embodiment of the present invention, the tubular member 41 may have a closed-end structure without an end cap.

The tubular member 41 is formed to accommodate the support bar 20 and the ultrasound probe 30. The tubular member 41 may include an ultrasound transmission window 43 through which ultrasound generated by the ultrasound probe 30 can pass. For example, the tubular member 41 may be formed of a metal material such as stainless steel or a plastic material. Although not shown in the drawings, a sealing film or sealing cover capable of fluid-tight sealing of the ultrasound transmission window 43 may be applied to the tubular member 41. The sealing film or sealing cover may be formed of a material that allows ultrasound generated by the ultrasound probe 30 to pass through while sealing the ultrasound transmission medium filled in the front space of the ultrasound probe 30.

According to an embodiment of the present invention, the ultrasound probe 30 is configured to be linearly movable. By configuring the support bar 20 to be linearly movable in the axial direction, the ultrasound probe 30 is configured to move linearly together with the support bar 20. The support bar 20 is fastened to the handle 10 such that it is movable in the axial direction. For example, the support bar 20 may be axially displaced by the driving force of a motor 61, such as a step motor, disposed in the handle 10. Referring to FIG. 3, the support bar 20 may be screw-coupled to a rotary element 63 connected to an output shaft 62 of the motor 61, in a state in which the rotary movement of the support bar 20 is restricted, such that the support bar 20 exhibits linear movement in response to rotation of the motor 61. When the motor 61 is operated and the rotary element 63 rotates, the support bar 20 screw-coupled to the rotary element 63 may perform axial linear motion. At this time, the ultrasound transmission window 43 of the sealing cover 40 may be formed to have a length longer than that of the ultrasound probe 30 so as to accommodate the linear movement of the ultrasound probe 30. With the linear movement mechanism of the ultrasound probe 30, the irradiation position of ultrasound can be changed without moving the handle 10, thereby enabling the formation of a linear thermal lesion.

The interior space of the sealing cover 40, in which the ultrasound probe 30 is disposed, is configured to be filled with water, which serves as the ultrasound transmission medium, and the filled water may be circulated. The supplied water fills the front of the ultrasound piezoelectric element 33, thereby enabling ultrasound propagation. According to an embodiment of the present invention, an ultrasound transmission medium supply tube, that is, a water supply tube 51, is provided to supply water to the space in which the ultrasound probe 30 is disposed. The water supply tube 51 may extend from the handle 10 to the ultrasound probe 30. For example, the water supply tube 51 may extend parallel to the support bar 20 within the sealing cover 40.

When the support bar 20 connected to the handle 10 performs linear movement, a sealing seal may be provided in the handle 10 to prevent the ultrasound transmission medium filled inside the sealing cover 40 from flowing into the interior of the handle 10, and the support bar 20 may be installed to pass through the sealing seal. In this case, the support bar 20 that passes through the sealing seal may be formed to have a different diameter so as to be in close contact with the inner peripheral surface of the sealing seal. The sealing seal may serve to support the support bar 20 and also function as a sealing member to prevent leakage of the ultrasound transmission medium.

The rear end of the water supply tube 51, which is fastened to the handle 10, may be fluidically connected to a supply pipe 81 that supplies water from an external source, and accordingly, water pumped by a water pump (not shown) may flow into the water supply tube 51 via the supply pipe 81. The water introduced through the water supply tube 51 may be configured to fill the space around the ultrasound probe 30 and the interior space of the sealing cover 40, and then be discharged. For example, the interior space of the sealing cover 40 may be fluidically connected to a drain pipe 83, such that water supplied through the water supply tube 51 fills the interior space of the sealing cover 40 and is then discharged through the drain pipe 83. Water drainage may be performed by a pump, and both water supply and drainage may be carried out simultaneously by the pumping action of the pump. In this manner, a circulation structure may be implemented in which water, as the ultrasound transmission medium, fills the distal portion inside the sealing cover 40 where the ultrasound probe 30 is disposed, flows through the gap between the sealing cover 40 and the housing 31, then fills the sealing cover 40, and is discharged from the proximal portion. Accordingly, ultrasound transmission and cooling functions can be effectively achieved. At this time, the housing 31 may have a shape in which both side surfaces are cut in parallel, so as to maximize the gap between the sealing cover 40 and the outer periphery of the housing 31.

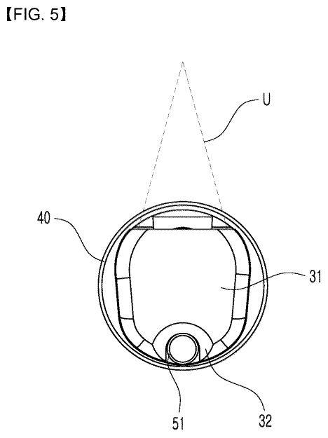

The water supply tube 51 is arranged to pass through the ultrasound probe 30. Referring to FIGS. 5 to 8, the end of the water supply tube 51 is formed to extend beyond the housing 31 of the ultrasound probe 30 and be exposed to the internal space located as close as possible to the distal end of the sealing cover 40. As a result, the supplied water is not delivered through the narrow space between the inner peripheral surface of the sealing cover 40 and the outer peripheral surface of the ultrasound probe 30 to reach the distal end of the sealing cover 40, but instead is directly supplied to the distal end portion of the internal space of the sealing cover 40 via the water supply tube 51 and through the ultrasound probe 30. This allows water to be easily delivered to the distal space inside the sealing cover 40 and to the space surrounding the ultrasound probe 30.

In particular, when the sealing cover 40 is mounted, air tends to remain in the space between the housing 31 of the ultrasound probe 30 and the distal end of the sealing cover 40. To remove this air, the water supply tube 51 is provided in the empty space between the housing 31 and the sealing cover 40. If configured otherwise, it becomes very difficult to remove the air trapped between the ultrasound probe 30 and the distal end of the sealing cover 40 and to fully fill the space with water.

In addition, due to the linear movement of the ultrasound probe 30, the space between the distal end of the sealing cover 40 and the housing 31 changes dynamically. In particular, when the housing 31 moves away from the distal end of the sealing cover 40, the newly created empty space must be filled with water in a short time. However, if the water supply tube 51 is not provided at the distal end of the sealing cover 40, it becomes extremely difficult to fill the space with water. Furthermore, if the space is not properly filled with water and the housing 31 of the ultrasound probe 30 moves closer again to the distal end, an adverse cycle may occur in which residual air in the space prevents proper filling with water.

Meanwhile, the housing 31 of the ultrasound probe 30 is provided with a recessed groove 32 that extends approximately in parallel with the longitudinal direction, and the water supply tube 51 is disposed in the recessed groove 32. By disposing the water supply tube 51 in the recessed groove 32, the water supply tube 51 can pass through the ultrasound probe 30 without increasing the overall cross-sectional area. As a result, an increase in the diameter of the sealing cover 40 in which the ultrasound probe 30 is placed can be prevented.

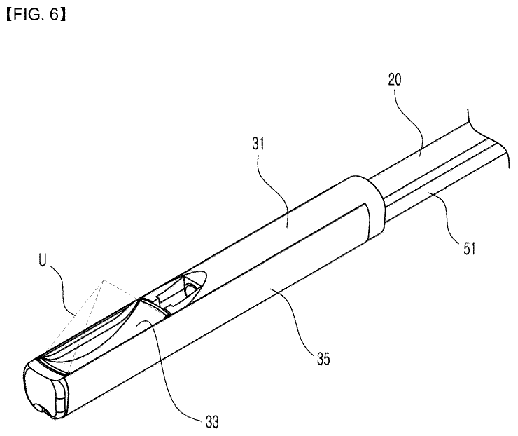

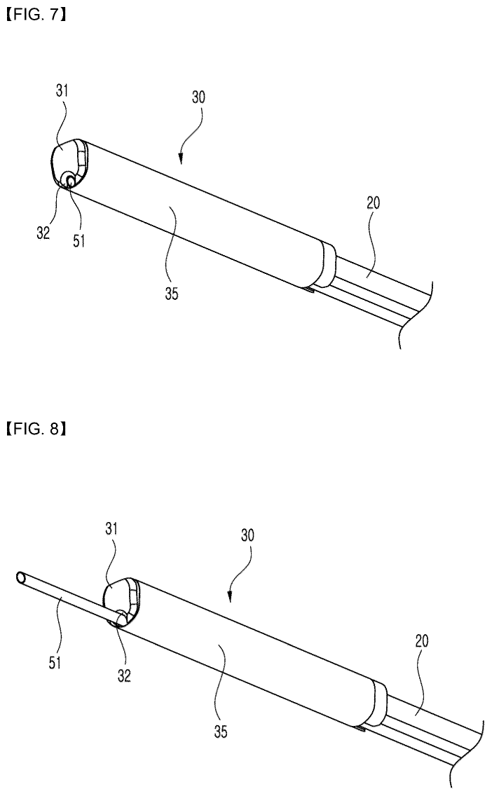

As described above, the ultrasound probe 30 is configured to be linearly movable, and for this purpose, the housing 31 of the ultrasound probe 30 is configured to move relative to the water supply tube 51 during the movement of the probe. That is, referring to FIGS. 7 and 8, when the support bar 20 and the ultrasound probe 30 move within the sealing cover 40, the water supply tube 51 should remain stationary. Since the water supply tube 51 is inserted into the recessed groove 32 of the housing 31, the water supply tube 51 can serve as a guide for the linear movement of the ultrasound probe 30. In this case, to prevent the housing 31 of the ultrasound probe 30 from becoming detached from the water supply tube 51, a fastening member, such as a fastening tape 35, may be used to fasten the housing 31 and the water supply tube 51 together. Here, the fastening tape 35 is fixed to the housing 31, while being configured to allow relative movement of the water supply tube 51.

FIG. 9 shows a cross-sectional view of an ultrasound piezoelectric element 130 according to another embodiment of the present invention. In this embodiment, a water supply tube 71 and a water discharge tube 73 are respectively provided. The water supply tube 71 is configured to directly supply water to the distal end space of the sealing cover 40, and the water discharge tube 73 is configured to discharge water from the distal end space of the sealing cover 40. The housing of the ultrasound piezoelectric element 130 includes a pair of recessed grooves 131 and 132 provided on both side surfaces, and the water supply tube 71 and the water discharge tube 73 are respectively inserted into the recessed grooves 131 and 132. By providing the water supply tube 71 and the water discharge tube 73 in communication with the distal end space of the sealing cover 40, smooth water supply and discharge can be achieved.

In another embodiment, a plurality of water supply tubes may be used for water supply. If the flow rate of water supplied through the water supply tube is low, a sufficient amount of water can be supplied by using multiple water supply tubes.

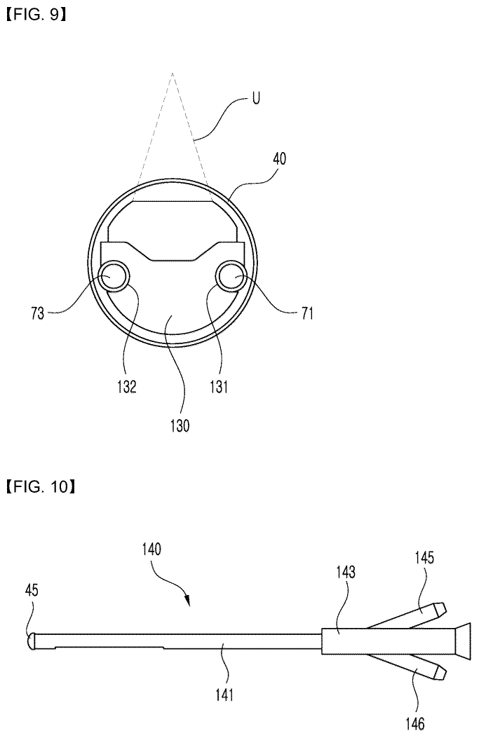

FIG. 10 shows a sealing cover 140 according to another embodiment of the present invention. The sealing cover 140 includes a pipe member 141, a connection member 143, and an end cap 45, in the same manner as the previously described embodiment. In this case, the water supply-type sealing cover 140 includes a water supply pipe connector 145 and a drainage pipe connector 146, which respectively form passages for water supply and drainage. Accordingly, water supply and drainage can be performed through the water supply pipe connector 145 and the drainage pipe connector 146, without passing through the handle 10.

While the embodiments of the present invention have been described above, the scope of the present invention is not limited thereto, and various modifications and alterations may be made by those skilled in the art without departing from the scope of the invention. Accordingly, all such modifications and equivalents should be construed as being included within the scope of the present invention.

1. An intracavitary ultrasound device comprising:
a support bar extending in a longitudinal direction;
an ultrasound probe supported by the support bar and configured to be insertable into a body cavity;
a sealing cover configured to surround at least a portion of the ultrasound probe and the support bar; and
an ultrasound transmission medium supply tube configured to supply an ultrasound transmission medium to a space in which the ultrasound probe is disposed,
wherein the ultrasound transmission medium supply tube is configured to supply the ultrasound transmission medium past the ultrasound probe to a distal end space of the sealing cover.

2. The intracavitary ultrasound device of claim 1, wherein the ultrasound probe comprises a housing, and an ultrasound piezoelectric element fixed to the housing, and
wherein the ultrasound transmission medium supply tube is configured such that an end thereof passes through the housing and is exposed to an inner space of the sealing cover.

3. The intracavitary ultrasound device of claim 2, wherein the housing comprises a recessed groove, and
wherein the ultrasound transmission medium supply tube is inserted into the recessed groove.

4. The intracavitary ultrasound device of claim 3, further comprising a fastening member that connects the housing and the ultrasound transmission medium supply tube to each other so as to prevent the ultrasound transmission medium supply tube from disengaging from the recessed groove of the housing.

5. The intracavitary ultrasound device of claim 4, wherein the fastening member is configured to fasten the housing and the ultrasound transmission medium supply tube to each other, such that relative movement between the housing and the ultrasound transmission medium supply tube is allowed, while preventing the ultrasound transmission medium supply tube from detaching from the housing.

6. The intracavitary ultrasound device of claim 1, wherein the ultrasound probe is disposed within the sealing cover in a linearly movable state.

7. The intracavitary ultrasound device of claim 6, wherein the ultrasound probe is configured to linearly move together with the support bar.

8. The intracavitary ultrasound device of claim 6, wherein the ultrasound transmission medium supply tube is configured to maintain a predetermined position during linear movement of the ultrasound probe and to function as a guide for the linear movement of the ultrasound probe.

9. The intracavitary ultrasound device of claim 1, further comprising an ultrasound transmission medium discharge tube configured to discharge the ultrasound transmission medium from the space in which the ultrasound probe is disposed.

10. The intracavitary ultrasound device of claim 9, wherein the ultrasound probe includes a housing and an ultrasound piezoelectric element fixed to the housing,
wherein the housing includes a pair of recessed grooves, and
wherein the ultrasound transmission medium supply tube and the ultrasound transmission medium discharge tube are respectively inserted into the pair of recessed grooves.

11. The intracavitary ultrasound device of claim 1, further comprising a handle to which the support bar is connected, wherein the ultrasound probe and the support bar are configured to move relative to the handle.

12. The intracavitary ultrasound device of claim 11, wherein the ultrasound transmission medium supplied into the sealing cover through the ultrasound transmission medium supply tube is configured to be discharged to the outside via either the inside or outside of the handle.

13. The intracavitary ultrasound device of claim 1, wherein the sealing cover comprises an ultrasound transmission medium inlet passage for the inflow of the ultrasound transmission medium, and an ultrasound transmission medium outlet passage for the discharge of the ultrasound transmission medium.

14. The intracavitary ultrasound device of claim 13, further comprising a handle to which the support bar is connected, wherein the sealing cover is configured to be detachable from the handle.

15. The intracavitary ultrasound device of claim 2, wherein the ultrasound probe is disposed within the sealing cover in a linearly movable state.

16. The intracavitary ultrasound device of claim 3, wherein the ultrasound probe is disposed within the sealing cover in a linearly movable state.

17. The intracavitary ultrasound device of claim 4, wherein the ultrasound probe is disposed within the sealing cover in a linearly movable state.

18. The intracavitary ultrasound device of claim 15 wherein the ultrasound probe is disposed within the sealing cover in a linearly movable state.
